(19) United States
(12) Patent Application Publication
(10) Pub. No.: US 2021/0379115 A1
(43) Pub. Date: Dec. 9, 2021

(54) HAIR REJUVENATION

(71) Applicant: HairClone Limited, Manchester Greater Manchester (GB)

(72) Inventors: Paul KEMP, Manchester Greater Manchester (GB); Vincent RONFARD, Manchester Greater Manchester (GB)

(21) Appl. No.: 17/286,019

(22) PCT Filed: Oct. 17, 2019
(86) PCT No.: PCT/GB2019/052971

§ 371 Date: Apr. 16, 2021

(30) Foreign Application Priority Data
Oct. 17, 2018 (GB) 1816902.9

(51) Int. Cl. A61K 35/36; C12N 5/071; A61P 17/14

(52) U.S. Cl. CPC A61K 35/36; C12N 5/0627; A61P 17/14; C12N 2500/14; C12N 2501/39; C12N 2500/40; C12N 2501/11

(57) ABSTRACT

The invention in relates in part to a method for rejuvenating hair follicles, the method comprising the steps of: (1) obtaining androgen non-inhibited cells from hair follicle tissue; (2) culturing the androgen non-inhibited cells to produce an expanded population of androgen-non-inhibited cells; and (3) implanting the expanded population of androgen non-inhibited cells proximal to miniaturised and/or miniaturising hair follicles.

### FIELD OF THE INVENTION

The invention relates to methods and preparations for hair rejuvenation.

### BACKGROUND OF THE INVENTION

The treatment of hair loss is a significantly unmet public health need. Hair loss can have a serious impact on the self-confidence and consequent quality of life of an individual. It has also been shown recently that male-pattern baldness was significantly associated with increased risk of squamous cell carcinoma and basal cell carcinoma particularly in the scalp region (Li et al., 2016, Int J Cancer 139(12): 2671-2678). Both males and females can suffer hair loss, with about 40% of men having noticeable hair loss by the age of 35 and about 80% of women experiencing noticeable hair loss by the age of 60. Androgenetic alopecia (AGA) is the most common form of hair loss in both men and women and is also known as male and female pattern baldness, respectively.

Hair grows from hair follicle structures which are multilayered, angled invaginations of the superficial epithelium. The hair follicle unit is composed of both mesenchymal and epithelium-derived cell populations in close association. The germinative epithelial cells of the hair bulb proliferate and differentiate to give rise to the mature hair shaft. The mesenchyme component consists of fibroblast-like cells that form the morphologic units known as the dermal papilla (DP), located at the base of the hair follicle unit, and the dermal sheath (DS) that exists around the outer limits of the epithelial hair follicle component. The DP is essential to hair follicle development and cycling. Biochemical signaling by DP cells controls the cell dynamics of the epidermal component and the overall physical size of the hair follicle.

The hair has other structures such as nerves, sebaceous glands, a blood supply, and an attached erector pili muscle that can alter the follicle's angle in the dermis. At the point at which this muscle attaches to the follicle itself there is a bulge region containing a reservoir of pluripotent epithelial stem cells called bulge cells.

Most body surfaces in humans apart from soles of the feet and palms of the hand, and lips contain hair follicles and it is estimated that an adult can have approximately 5 million hair follicles of which around 100,000 are on the scalp. The density of hair follicles varies with region from around 50 per cm² on the thigh to up to 600 per cm² on the scalp. As well as hair follicle density variations have shaft diameter and length is also very variable with fine vellus hairs approximately 10-30 μm in diameter covering the forehead with nearby “terminal hairs” that are approximately 200 μm in diameter covering the scalp.

The development of a hair follicle begins when the basal cells of the epidermis form a visible hair placode and dermal fibroblasts begin to aggregate under the placode and differentiate to form a spherical DP. The epidermal cells form a multi-layered and elongated column called the hair peg which then thickens at the lower end to form a hair bulb which then encloses the elongated DP and the developing follicle grows down into the dermis. Differentiation of the epithelial cells in the hair peg into defined layers continues and a hair shaft is produced. Finally, this hair shaft protrudes from the skin surface and the hair follicle reaches its maximal length. The morphogenesis of a hair follicle thus requires intensive communications and joint development of the epidermal and dermal compartments.

It has long been known that implantation of freshly isolated DP cells into adult skin can repeat this developmental process and induce the formation of new hair follicles from undifferentiated, inter-follicular epidermis. When this was first discovered it opened the possibility that DP cells could be used to induce brand new hair follicles.

Four methods have been described that are able to support expansion of cell number while also maintaining the hair inductive potential of DP cells to create new hair follicles. In a first method, conditioned medium (CM) collected from mammalian epidermal cells (keratinocytes), or co-culture with keratinocytes, were shown to support both expansion of DP cell number and maintenance of the hair inductive phenotype over several culture passages. This first method is described in U.S. Pat. No. 5,851,831 and in Jahoda et al. (1998, J. Invest. Dermatol. 111: 767-75). A second method describes the culture of DP cells in the presence of an increased level of wnt protein or an agent that mimics the effects of wnt-promoted signal transduction (see WO01/74164 and Kishimoto et al., 2002, Genes & Development 14: 1181-85). In the second method, a wnt protein or a functional fragment or analogue thereof is added to the culture medium as a purified product, or by expressing a recombinant protein in producer cells and providing the with protein in medium conditioned by the growth of wnt producer cells, or by co-culture with producer cells. A third method relates to culturing the hair inductive cells in a culture medium comprising prostate epithelial cells and medium conditioned by prostate epithelial cells. A fourth method involves culturing DP cells in 3D hanging drops (see U.S. Pat. Nos. 9,550,976, 9,109,204, and Higgins et al., 2013, Proc Natl Acad Sci USA 110(49): 19679-88).

However it has also been shown that DP cells change their transcription profile and lose their hair inductive potential when grown in standard 2D culture which limits their potential to treat AGA. Higgins et al. (2013, supra) describes partial restoration of hair follicle inductivity in DP cells in three-dimensional (3D) cultures, leading to hair follicle induction. U.S. Pat. No. 9,109,204 describes methods of aggregating DP cells for promoting the formation of hair follicles ex vivo, for example in amputated hair follicles.

Current hair loss therapies include oral and topical treatments and hair transplantation surgery. The currently available oral and topical treatments for hair loss must be taken lifelong and are only effective during the early stages of hair loss. Only two therapeutic agents have been approved currently by the US Food and Drug Administration (FDA) and European Medicines Agency (EMA) for the treatment of androgenetic alopecia (AGA): topical minoxidil and oral finasteride. Minoxidil can slow the progression of hair loss and partially restore hair but is expensive and effective in only a small proportion of males and females. Even for those individuals who respond, treatment must be maintained in order to sustain the effect. Minoxidil can also cause irritation and/or allergic reactions at the site of application. Finasteride is only effective in men, and then in only a proportion of men. Finasteride may also cause long-term sexual side effects such as decreased libido and erectile dysfunction. Any beneficial effects on hair growth are lost within 6 to 12 months of discontinuing treatment.

Hair loss may alternatively be treated by transplanting hair follicles from regions that are expected to be unaffected by AGA to areas where hair loss is occurring (for example, frontal, mid scalp and crown region of the scalp). However, there are limitations on the numbers of follicles available for transplantation and the subject is left with the same number of hairs following transplantation, as the hair is merely distributed from high to low hair density regions. Also, in men just beginning to show the effects of AGA hair transplantation is often not suitable as the boundary of affected and unaffected regions are not yet clear. The effectiveness of hair transplantation is therefore limited by the patients who are suitable and the number of available donor hairs. However, despite the limits of this surgery, worldwide in 2016 there were over 500,000 surgical hair restoration procedures, creating a US$ 4.1 billion market.

A 2015 survey of the International Society of Hair Restoration Surgeons (ISHRS), found that the most common complaint of hair transplant patients was either that the resultant hair density was less than expected (57% of all complaints) or that hair loss occurred post-transplant (39% of complaints). Hair transplantation is considered “hair restoration”, as it can only take place when the boundary of hair loss is properly understood in a particular patient. Consequently, approximately 80% of male transplant patients are over 30 years old and have already suffered significant hair loss.

### SUMMARY OF THE INVENTION

The invention in general relates to a method of cellular rejuvenation of hair follicles that are miniaturising, for example as a result of male or female androgenic alopecia, by introducing androgen non-inhibited cells into affected regions whereby the androgen non-inhibited cells at least partially replace the lost androgen-inhibited cells thereby increasing hair shaft diameter and apparent hair density thus at least partly reversing the appearance of hair loss.

In one aspect, the invention is directed to a method for rejuvenating hair follicles, the method comprising the steps of (1) obtaining androgen non-inhibited cells from hair follicle tissue; (2) culturing the androgen non-inhibited cells obtained in step (1) under conditions suitable for proliferation, to produce an expanded population of androgen non-inhibited cells; and (3) implanting the expanded population of androgen non-inhibited cells produced in step (2) proximal to miniaturised and/or miniaturising hair follicles, thereby rejuvenating the miniaturised and/or miniaturising hair follicles.

The androgen non-inhibited cells may be androgen insensitive cells, for example wherein the hair follicle tissue is scalp hair follicle tissue.

Alternatively, the androgen non-inhibited cells may be androgen stimulated cells, for example wherein the hair follicle tissue is beard hair follicle tissue, chest hair follicle tissue, axilla hair follicle tissue and/or pubic hair follicle tissue.

Optionally, the androgen non-inhibited cells in the method of the invention comprise or consist of dermal papilla (DP) cells. DP cells have been shown to possess particularly potent rejuvenation capabilities.

The androgen non-inhibited cells implanted in step (3) of method of the invention may rejuvenate the miniaturised and/or miniaturising hair follicles by reactivating androgen-sensitive cells of the miniaturised and/or miniaturising hair follicles, and/or replacing androgen-sensitive cells in the miniaturised and/or miniaturising hair follicles. Other mechanisms of hair rejuvenation may also occur.

The hair follicle tissue may be extracted mechanically, for example using follicle unit extraction (FUE). Alternatively, hair follicle tissue may be obtained using follicular unit transplantation (FUT).

The method of the invention may include the step of culturing the androgen non-inhibited cells to expand the number of androgen non-inhibited cells by at least about two- to twenty-fold, or by at least about 500- to 1000-fold, or by at least one to six population doublings. Expanding the number of androgen non-inhibited cells increases the number of cells for implantation.

Step (1) and/or (2) of the method may include a step of selecting and/or isolating (for example, by sorting) androgen non-inhibited cells from a mixed population of cells in the hair follicle tissue, for example using one or more biomarkers. Selecting or sorting the cells removes unwanted androgen-sensitive cells from the cell population.

In the method of the invention, the androgen non-inhibited cells may be obtained from a subject in step (1) of the method and after culturing in step (2) of method implanted into same subject in step (3) of method, i.e. the implanted cells are autologous. Autologous cells will be more likely to be tolerated by the host tissue and/or be particular effective at hair rejuvenation for a particular individual. However, the use of allogeneic cells is also contemplated in the present invention.

In another aspect, the invention relates to a composition comprising an in vitro-expanded population of androgen non-inhibited cells for rejuvenating hair. The androgen non-inhibited cells may have any of the features as those used in the method of the invention.

The cells of the composition may be from an autologous or allogeneic origin, for example relative to a subject treated with the cells.

The composition may be formulated and administered as an injection, for example by a physician or a non-physician medical technician.

The composition may be used in a method of treatment, for example a cosmetic method of treatment.

The composition may be used in a method for the rejuvenation of hair growth and/or for the retardation of hair loss.

The composition may be used in the manufacture of a medicament for the rejuvenation of hair growth and/or for the retardation of hair loss.

The composition may be used for application to a subject having alopecia, such as androgenic alopecia.

The composition of the invention may be used in a system for analysing hair follicle cells and/or for testing a cosmetic or pharmaceutical agent.

### DETAILED DESCRIPTION OF THE INVENTION

### Abbreviations

3D three-dimensional
5α-DHT 5-alpha-dihydrotestosterone
AGA androgenetic alopecia
DHT dihydrotestosterone
DP dermal papilla
EMA European Medicines Agency
FDA Food and Drug Administration
FUE follicle unit extraction
FUT follicular unit transplantation.

### Terminology

Before describing the present invention in detail, it is to be understood that this invention is not limited to specific compositions or process steps.

A cosmetic method is defined as a method for improving the appearance of an individual. Unless defined otherwise, all technical and scientific terms used herein have the same meaning as commonly understood by one of ordinary skill in the art to which this invention is related. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

Standard techniques are typically used for tissue culture, including for proliferating cells in conditions suitable for proliferation. Suitable methods, reagents and condition for culturing cells obtained from hair follicle tissue are provided in the specific Examples. Enzymatic reactions and purification techniques may be performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Methods suitable for obtaining androgen non-inhibited cells from tissue and implanting a population of cells are provided in the specific Examples. Androgen non-inhibited cells may be androgen insensitive cells or androgen stimulated cells. Androgen sensitive cells can be obtained, for example, from non-balding regions of the scalp. Androgen stimulated cells may be obtained, for example, from beard, chest, axilla or pubic regions.

As utilized in accordance with the present disclosure, the following terms, unless otherwise indicated, shall be understood to have the following meanings:

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to ±10% (and values there between, e.g., ±0.5%, ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%). The use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%).

### Invention Aspects

Hairs show repeated cycles of growth and rest. The three stages of this cycle are the anagen, catagen, and telogen phases. Although in some animals, these cycles are synchronized, each strand of hair on the human body is asynchronous and at its own stage of this cycle. The anagen phase is the growth phase and can last from a few months to a few years. The longer the hair stays in the anagen phase, the longer it will grow. About 85% of the hairs on a human's scalp are in this anagen phase at any given time. This growth phase is then followed by the catagen phase which lasts about two weeks, during which time the cellular structure of the hair follicle degenerates to around ⅙ its original length. While hair is not actively growing during this phase, the hair shaft is still anchored in place in the skin. Following catagen the hair follicle rests in the telogen phase in which the follicle remains dormant for one to four months. At some point, a new cycle is initiated and the follicle structure completely regenerates again. The hair base will break free from the root and this old hair shaft will be shed. Within two weeks a new hair shaft will begin to emerge as the next anagen phase begins. The new hair shaft is normally a similar size and structure to that produced in the previous cycle. In an average human life span a scalp hair will go through approximately 12 of these cycles. This cycling is known to be androgen dependent and changes in androgen levels can result in significant changes in hair shaft diameter and length in specific regions between one cycle and the next. During puberty for example changes in androgen levels can cause increase in hair shaft diameter in certain regions. However, androgens can also cause a reduction of hair shaft diameter and a miniaturisation of the hair follicle itself through progressive cycles and it is this miniaturising during conditions such as AGA which is most notable on the scalp.

Not all hair follicles respond to hormones in this way and AGA only affects certain androgen-sensitive hair follicles. On the scalp itself this is most notable in men as male pattern baldness, whereby hairs miniaturised in a progressive, temporal pattern beginning at the temples and spreading back across the scalp. This miniaturisation of the diameter of hair follicles from terminal to vellus state occurs over a number of cycles. Moreover during this miniaturising process, the length of time of the hair follicle remains in the anagen or growth phase of the follicle decreases significantly, so the balding follicle spends a greater proportion of its follicle cycle in telogen, the non-growing stage and the hair shaft length as well as diameter decreases.

Hair follicle size and the resulting hair shaft diameter is determined by its mesenchymal component and it has been shown that human follicle DP miniaturisation is a direct result of reduction in papilla cell numbers in the hair follicle over time and it appears that this decrease occurs between anagen phases and generation of the next hair shaft.

Human DP cells from different body sites differ in their responsiveness to androgens. Particularly, androgens (for example, dihydrotestosterone [DHT] or 5α-dihydrotestosterone [5α-DHT]) stimulate hair follicle growth for example in the axilla, pubic area and male beard but cause follicle miniaturisation in the scalp in patient suffering from AGA. Some hair follicles are insensitive to androgens and remain approximately the same size through life. Human DP cells can therefore be categorised as androgen inhibited (such as those affected by AGA) and androgen non-inhibited. These androgen non-inhibited DP cells can further be subdivided into androgen stimulated (such as in the male beard region) or androgen-insensitive (such as those hairs not affected by AGA).

In one aspect, the invention is directed to a method for rejuvenating hair follicles, the method comprising the steps of (1) obtaining androgen non-inhibited cells from hair follicle tissue; (2) culturing the androgen non-inhibited cells obtained in step (1) under conditions suitable for proliferation, to produce an expanded population of androgen non-inhibited cells; and (3) implanting the expanded population of androgen non-inhibited cells produced in step (2) proximal to miniaturised and/or miniaturising hair follicles, thereby rejuvenating the miniaturised and/or miniaturising hair follicles.

The invention may involve replacing lost androgen sensitive DP cells in the miniaturised and/or miniaturising hair follicles with androgen non-inhibited DP cells, such as androgen insensitive DP cells, as well as increasing the hair shaft diameter. This could be considered as changing the character of the hair follicle itself over subsequent hair cycles, in effect converting a hair follicle affected by AGA into a hair follicle unaffected by AGA.

The current invention differs from prior art disclosures in part because it relates to the use of specific androgen non-inhibited cells to rejuvenate hair follicles that are miniaturising because of the reduction of the number of androgen-sensitive cells, thereby causing an increase in the diameter of the hair follicles.

The androgen non-inhibited cells may be androgen insensitive cells. These cells may be obtained from scalp hair follicle tissue. The androgen non-inhibited cells may be androgen stimulated cells, and may be obtained from beard hair follicle tissue, chest hair follicle tissue, axilla hair follicle tissue and/or pubic hair follicle tissue.

The androgen insensitive cells may be obtained from male subjects, for example from the back of the head.

Optionally, the androgen non-inhibited cells in the method of the invention comprise or consist of DP cells. DP cells have been shown to possess particularly potent rejuvenation capabilities.

The androgen non-inhibited cells implanted in step (3) of method of the invention may rejuvenate the miniaturised and/or miniaturising hair follicles by reactivating androgen-v cells of the miniaturised and/or miniaturising hair follicles, and/or replacing androgen-sensitive cells in the miniaturised and/or miniaturising hair follicles. Other mechanisms of hair rejuvenation may also occur.

Prior art methods relating to the culturing of DP cells described above require the DP cells to maintain their ability to induce new follicles to form by the interaction of the cultured DP cells with the recipient's keratinocytes. However, regulating the number of DP cells in existing miniaturising hair follicles represents an alternative strategy for preventing hair loss by repairing and rejuvenating these declining follicles. Experimental analysis of DP cell function has shown that, under appropriate conditions, implanted mouse DP cells can are able to “home” to the DP region, form chimeric DP and influence hair shaft diameter (see McElwee et al., 2003, Invest Dermatol 121: 1267-1275). It is suggested that androgen non-inhibited cells implanted in step (3) of the method may act in the same way.

The hair follicle tissue used in the method of the invention may be obtained from a body region known or expected to comprise androgen non-inhibited cells. This advantageously provides for efficient collection of androgen non-inhibited cells.

However, although androgen non-inhibited cells from hair follicle tissue are generally found in particular regions, in certain embodiments location alone cannot be used to predict the androgen responsiveness of the follicles. For example, while androgen sensitive cells can be found at the back of the scalp in males affected by AGA, in females the hairloss pattern may be more diffuse. Screening of cells obtained from scalp hair follicle tissue for androgen sensitivity, androgen non-sensitivity and/or androgen stimulation may thus comprise a further step in the method of the invention. In this way, only or predominantly androgen non-inhibited cells may be cultured in step (2) of the method or implanted in step (3) of the method.

The hair follicle tissue may be extracted mechanically, for example using follicle unit extraction (FUE). Alternatively, hair follicle tissue may be obtained using follicular unit transplantation (FUT).

In one embodiment of the invention, obtaining androgen non-inhibited cells from hair follicle tissue involves obtaining androgen non-inhibited cells from a suspension of hair follicle tissue cells by antibody-assisted selection. This provides marker determined selection of the androgen non-inhibited cells.

The method of the invention may include the step of culturing the androgen non-inhibited cells to expand the number of androgen non-inhibited cells by at least about two- to twenty-fold, or by at least about 500- to 1000-fold, or by at least one to six population doublings. Expanding the number of androgen non-inhibited cells increases the number of cells for implantation.

Step (1) and/or (2) of the method may include a step of selecting and/or isolating (for example, by sorting) androgen non-inhibited cells from a mixed population of cells in the hair follicle tissue, for example using one or more biomarkers. Selecting or sorting the cells removes unwanted androgen-sensitive cells from the cell population.

In one embodiment of the invention, the level of expression of one or two or more (for example, three, four, five, six, seven, eight, nine or ten or more) genes is used as a biomarker of androgen insensitivity to identify and/or select androgen non-inhibited cells. The one or two or more genes may be selected from the group consisting of STX17 anti-sense RNA 1; prostaglandin I2 (prostacyclin) synthase; calmegin; glypican 6; integrin beta 8; collagen, type X, alpha 1; reelin; carbonic anhydrase XIII; DEP domain containing MTOR-interacting protein; and HAUS augmin like complex subunit 6.

The above-listed genes have been found to be expressed in higher levels in androgen non-inhibited DP cells but not expressed or expressed only in low levels in androgen-sensitive DP cells. The genes provide for efficient and accurate identification and selection of androgen non-inhibited cells.

Additional or alternative genes whose expression or non-expression can be used a biomarker for androgen non-inhibited cells or androgen-inhibited cells are shown in Example 4 below (specifically, in Table 1 and Lists A-D). The biomarker may for example be STON1 (Stonin-1), CYB5R3 (encoding Cytochrome B5 Reductase 3) and/or SRD5A1 (encoding Steroid 5 alpha-reductase 1).

Expression or non-expression of appropriate control genes known in the art can be tested for comparative purposes.

The steps (1) and/or (2) of the method of the invention may comprise selecting or sorting the cells for androgen non-inhibited cells using the biomarker. Selecting or sorting the cells removes unwanted androgen-sensitive cells from the cell population.

The androgen of the method of the invention may be dihydrotestosterone (DHT) and/or the androgen non-inhibited cells may be human.

In the method of the invention, the androgen non-inhibited cells may be obtained from a subject in step (1) of the method and after culturing in step (2) of method implanted into same subject in step (3) of method, i.e. the implanted cells are autologous. Autologous cells will be more likely to be tolerated by the host tissue and/or be particular effective at hair rejuvenation for a particular individual.

In another aspect, the invention relates to a composition comprising an in vitro-expanded population of androgen non-inhibited cells for rejuvenating hair. The androgen non-inhibited cells may have any of the features as those used in the method of the invention.

The cells of the composition may be from an autologous or allogeneic origin, for example relative to a subject treated with the cells.

The composition may be formulated and administered as an injection, for example by a physician or a non-physician medical technician.

The composition may be used in a method of treatment, for example a cosmetic method of treatment.

The composition may be used in a method for the rejuvenation of hair growth and/or for the retardation of hair loss.

The composition may be used in the manufacture of a medicament for the rejuvenation of hair growth and/or for the retardation of hair loss.

The composition may be used for application to a subject having alopecia, such as androgenic alopecia. This use may be entirely cosmetic.

The composition of the invention may be used in a system for analysing hair follicle cells and/or for testing a cosmetic or pharmaceutical agent.

The entirety of each patent, patent application, publication and document referenced herein hereby is incorporated by reference to the same extent as if each individual publication, patent or patent application was specifically and individually indicated to be incorporated herein by reference. Citation of the above patents, patent applications, publications and documents is not an admission that any of the foregoing is pertinent prior art, nor does it constitute any admission as to the contents or date of these publications or documents.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

Further aspects, embodiments, features, and the like regarding the invention as disclosed herein are provided in additional detail below.

### EXAMPLES

The following examples, including the experiments conducted and results achieved, are given so as to illustrate the practice of this invention. They are not intended to limit or define the entire scope of this invention.

### Example 1

### Obtaining Samples of Follicles Using Follicular Unit Transplantation (FUT)

Hair follicle cells were obtained using the FUT surgical procedure (see Bernstein & Rassman, 2005, Dermatologic Clinics 23(3): 393-414). A small strip of tissue was removed from androgen non-inhibited region of the body such as the back of the head from which the donor hair follicles can be extracted. Then, individual hair follicles were harvested from the strip. Hair follicles were stored frozen for future use or further micro dissected in order to be cultured (see also Example 3). Hair follicle units are full thickness and therefore have an epidermis, dermis and dermal adipose tissue.

Each follicle unit was trimmed from any adherent adipose or connective tissue surrounding the lower section of the follicle to expose the end bulb located at the base of the follicle. Using a pair of scissors, the follicle was transected through the matrix just above the papilla to isolate the end bulb. After cutting the end bulbs, the dermal papilla (DP) were isolated from the capsule of the dermal sheath as described in Higgins et al. (2017, Br. J. Dermatol. 176(5): 1259-1269). In more detail, the follicles were each transected just above the dermal papilla in order to isolate the end bulbs. The end bulbs were held in place by pressing a 27 G needle against the cut top and holding it to the petri dish. A second 27 G needle was held in the other hand and pushed gently through the round bottom of the end bulb to invert the structure and expose the DP. The DP surrounded by its glassy membrane was removed using the needle's sharp edge and transferred to a 50 μl drop of collagenase in a fresh petri dish. Each DP was dissected in a similar fashion and placed in its own 50 uL drop of collagenase and incubated for 30 min. This allows the DP to adhere to the petri dish but doesn't dissolve the glassy membrane. Using 2 ml of DMEM supplemented with 10% FBS, 2 mM L-glutamine (Invitrogen, cat. no. 25030), DP were then carefully added to each dish taking care not to dislodge the DP and the dishes cultured without moving them for 10 days at 37° C. in 10% CO₂ incubator. The DP cells were then serially passaged as described in Example 3.

In alternative embodiments, cells are isolated from the hair follicle unit by mechanical and/or enzymatic treatment. Fibroblasts, keratinocytes and adipocytes are readily isolated by disaggregating FUT-obtained samples. Disaggregation may be readily accomplished using several techniques known to those skilled in the art. Examples of such techniques include, but are not limited to, mechanical disaggregation and/or treatment with digestive enzymes and/or chelating agents that weaken the connections between neighboring cells, thereby making it possible to disperse the tissue into a suspension of individual cells without appreciable cell breakage. Mechanical disruption can be accomplished by a number of methods including, but not limited to, the use of shakers or spinners. In one embodiment, enzymatic dissociation is accomplished by mincing the tissue and treating the minced tissue with trypsin. Any of a number of digestive enzymes, either alone or in combination can be used—for example trypsin, chymotrypsin, collagenase, elastase, hyaluronidase, DNase, pronase, and/or dispase. Once the source tissue are reduced to a suspension of individual cells, cells are cultured in selective media to grow keratinocytes, fibroblasts, adipose derived cells, mesenchymal cell types or any other cell type.

### Example 2

### Obtaining Sample of Follicles by Follicular Unit Extraction (FUE)

In FUE, each follicular unit was individually taken directly from an androgen non-inhibited region of the body (e.g. back of the scalp, the beard region or other area of expected androgen non-inhibited follicles) with no strip of tissue being removed. The same procedures as described in Example 1 was used to isolate the DP for analysis or culture (see Example 3).

### Example 3

### Culture of Androgen Non-Inhibited Cells

Androgen non-inhibited cells were isolated from a hair follicle biopsy of a non-balding (androgen-insensitive) area obtained from FUT (as described in Example 1). Androgen non-inhibited cells were serially amplified in culture between 2 to 1000 times in DMEM supplemented with 10% FBS, 2 mM L-glutamine (Invitrogen, cat. no.25030), 1% ABAM. Medium was changed every 2-3 days. When the culture reached confluency, cells were either frozen, amplified or used in a composition to be applied on a patient. Once the cells were growing well, they were passaged using standard cell culture techniques subculturing at a 1:2 to 1:5 ratio.

The same method as described above was used for culture of androgen inhibited cells obtained in Example 2.

In another embodiment, androgen non-inhibited cells are amplified in culture between 2 to 100 times (at least). Keratinocytes are obtained from the explantation of hair follicles on a microporous membrane of a cell culture insert that carried on its undersurface a preformed feeder layer made of 10⁵ postmitotic human dermal fibroblast as described by Limat et al. (1989, J. Invest. Dermatol. 92(5): 758-62). The culture medium consists of Dulbecco's Modified Eagle Medium (DMEM; Invitrogen, cat. no. 11960)/F12 (3:1) supplemented with 10% fetal bovine serum (FBS; Invitrogen), 10 ng of epidermal growth factor per ml, 0.4 μg of hydrocortisone per ml, 0.1 nM cholera toxin, 0.135 mM adenine, and 2 nM triiodothyronine final Ca²⁺ concentration 1.5 nM. Within about 2 weeks, keratinocytes are expanded and reached confluence. They are dissociated with trypsin/EDTA 0.1%/0.02%, then checked for viability. They can be stored frozen or used in a preparation to treat patients. One hair follicle outgrowth covers 1-2 cm² of culture surface area in about 2 weeks. These cells can be passaged in the same medium for further expansion if necessary. Fibroblast are isolated from explant outgrowth of hair follicle or from single suspension after enzymatic treatment of the hair follicle. In both cases, DMEM supplemented with 10% FBS, 2 mM L-glutamine (Invitrogen, cat. no. 25030) and 0.1 mM (0.7 μl/100 ml final media volume) 2-mercaptoethanol (Sigma, cat. no. M7522) is used a growth medium to amplify the cells. Medium is changed every 2-3 days. When the culture reaches confluency, cells are either frozen, amplified or used in a composition to be applied on a patient. The same method can be used for culture of androgen-inhibited cells.

### Example 4

### Characterisation of Androgen Non-Inhibited DPs

A sample of androgen non-inhibited DP cells isolated as described in Example 1 and cultured as described in Example 3 was tested for gene expression characteristics of androgen non-inhibited cells. This was compared with gene expression characteristics of cultured androgen-inhibited DP cells obtained as described in Example 2 from follicles growing in the margins of balding regions and cultured in the same conditions described in Example 3.

For this transcriptome analysis, RNA was collected using the RNeasy Plus Micro Kit (Qiagen). RNA was used to synthesize first-strand complementary DNA (cDNA) using Nugen Ovation V2. This was converted to double-stranded cDNA, and used as a template for in vitro transcription to generate cRNA using the Nugen Encore Biotin Module. The cRNA was then transferred for hybridization and scanning using an Affymetrix U133 Plus 2.0 Array (Thermo Fisher Scientific Catalog no. 900466). Differential gene expression analysis was performed using Transcriptome Analysis Console (TAC) software (ThermoFisher Scientific). Expression or non-expression of appropriate control genes known in the art was tested for comparative purposes.

The biomarkers (gene transcripts, including non-protein encoding mRNA transcripts, collectively referred to herein as “genes”) showing the greatest level of differential expression between androgen-inhibited cells and androgen non-inhibited cells are shown in Table 1 below.

TABLE 1
Most differentially expressed genes in cultured androgen-inhibited
cells and androgen non-inhibited cells

| | | | Androgen- | Androgen |
|---|---|---|---|---|
| | Affymetrix | Gene | inhibited | non-inhibited |
| Gene | ID | Symbol | cells | cells |
| STX17 antisense RNA 1 | 1558688_at | STX17-AS1 | − | + |
| prostaglandin I2 (prostacyclin) | 208131_s_at | PTGIS | − | + |
| synthase | | | | |
| calmegin | 205830_at | CLGN | − | + |
| glypican 6 | 227059_at | GPC6 | − | + |
| integrin beta 8 | 226189_at | ITGB8 | − | + |
| collagen, type X, alpha 1 | 217428_s_at | COL10A1 | − | + |
| reelin | 205923_at | RELN | − | + |
| carbonic anhydrase XIII | 231270_at | CA13 | − | + |
| DEP domain containing MTOR- | 238623_at | DEPTOR | − | + |
| interacting protein | | | | |
| HAUS augmin like complex | 218858_at | HAUS6 | − | + |
| subunit 6 | | | | |
| cerebellar degeneration related | 207276_at | CDR1 | + | − |
| protein 1 | | | | |
| collagen, type VI, alpha 1 | 212939_at | COL6A1 | + | − |
| ribosomal protein L37a | 214041_x_at | RPL37A | + | − |
| transmembrane p24 trafficking | 204426_at | TMED2 | + | − |
| protein 2 | | | | |
| transmembrane protein with | 224321_at | TMEFF2 | + | − |
| EGF-like and two follistatin-like | | | | |
| domains 2 | | | | |
| dimethylarginine | 1553565_s_at | DDAH1 | + | − |
| dimethylaminohydrolase 1 | | | | |
| parathymosin | 218045_x_at | PTMS | + | − |
| transmembrane p24 trafficking | 204427_s_at | TMED2 | + | − |
| protein 2 | | | | |
| TAF13 RNA polymerase II, | 205966_at | TAF13 | + | − |
| TATA box binding protein | | | | |
| (TBP)-associated factor, 18 kDa | | | | |
| muscleblind like splicing | 1555594_a_at | MBNL1 | + | − |
| regulator 1 | | | | |

Table 1 shows the genes most highly expressed in one cell type (“+”) compared with the other cell type (“−”).

Genes expressed more than 10-fold higher in androgen non-inhibited cells compared with androgen inhibited cells (in addition to those shown in Table 1 above) are shown below (List A):

nuclear factor I/B; Rap guanine nucleotide exchange factor 6; CUGBP, Elav-like family member 2; receptor tyrosine kinase-like orphan receptor 1; RAB27B, member RAS oncogene family; metastasis suppressor 1; myelin basic protein; caspase recruitment domain family, member 10; dedicator of cytokinesis 4; transcription factor 7-like 2 (T-cell specific, HMG-box); DEAD (Asp-Glu-Ala-Asp) box polypeptide 52; GTPase, IMAP family member 2; spalt-like transcription factor 2; inositol 1,4,5-trisphosphate receptor, type 3; family with sequence similarity 132, member B, high mobility group AT-hook 1; kazrin, periplakin interacting protein; GDNF-inducible zinc finger protein 1; glutathione peroxidase 3; secretogranin II; chromosome X open reading frame 23; formin homology 2 domain containing 3; tetraspanin 12; protein tyrosine phosphatase, receptor type, E; coagulation factor II (thrombin) receptor-like 2; ribonuclease P/MRP 40 kDa subunit; leucine rich repeat containing 32; latexin; zinc finger protein 184; brain and acute leukemia, cytoplasmic; neural cell adhesion molecule 1; branched chain amino-acid transaminase 1, cytosolic; calcium/calmodulin-dependent protein kinase kinase 2, beta; mitochondrial calcium uptake family, member 3; homer scaffolding protein 2; NADH dehydrogenase (ubiquinone) Fe-S protein 1, 75 kDa (NADH-coenzyme Q reductase); follistatin like 1; vitrin; lysine (K)-specific demethylase 4B; long intergenic non-protein coding RNA 520; nuclear factor I/B; kelch-like family member 20; claudin 1; Ras-related GTP binding D; basonuclin 1; plexin domain containing 1; transforming growth factor beta receptor III; adriamycin resistance-associated; DnaJ (Hsp40) homolog, subfamily C, member 12; chordin-like 1; and sodium channel, voltage gated, type II alpha subunit.

Genes expressed more than 10-fold higher in androgen-inhibited cells compared with androgen non-inhibited cells (in addition to those shown in Table 1 above) are shown below (List B):

AHNAK nucleoprotein; serpin peptidase inhibitor, Glade E (nexin, plasminogen activator inhibitor type 1), member 2; lysyl oxidase; extended synaptotagmin-like protein 2; EGF containing fibulin-like extracellular matrix protein 1; collagen, type VI, alpha 1; protein tyrosine phosphatase, non-receptor type 11; collagen, type XII, alpha 1; eukaryotic translation elongation factor 1 delta (guanine nucleotide exchange protein); metastasis suppressor 1-like; talin 1; TSC22 domain family, member 2; insulin like growth factor binding protein 5; discoidin, CUB and LCCL domain containing 2; transforming growth factor beta 2; synaptopodin 2; dehydrogenase/reductase (SDR family) member 7; consortin, connexin sorting protein; microtubule associated monooxygenase, calponin and LIM domain containing 2; mitochondrial ribosomal protein L41; ribosomal protein S19; nuclear casein kinase and cyclin-dependent kinase substrate 1; collagen, type VI, alpha 1; FK506 binding protein 1A; cyclin K; WAS protein family, member 2; tousled-like kinase 1; MDM2 proto-oncogene, E3 ubiquitin protein ligase; spectrin, beta, non-erythrocytic 1; heat shock protein, alpha-crystallin-related, B6; RAB5A, member RAS oncogene family; caldesmon 1; glycogen synthase kinase 3 beta; homeodomain interacting protein kinase 3; nuclear factor I/X (CCAAT-binding transcription factor); polypyrimidine tract binding protein 3; charged multivesicular body protein 4B; PTPRF interacting protein, binding protein 1 (liprin beta 1); WD repeat and SOLS box containing 1; DnaJ (Hsp40) homolog, subfamily B, member 4; alkB homolog 7; GTPase activating protein (SH3 domain) binding protein 1; microtubule associated protein 1A; CKLF-like MARVEL transmembrane domain containing 6; MDM2 proto-oncogene, E3 ubiquitin protein ligase; t-complex 11, testis-specific-like 2; Rho GDP dissociation inhibitor (GDI) alpha; WW domain containing adaptor with coiled-coil; F-box protein 32; AT rich interactive domain 5B (MRF1-like); GrpE-like 2, mitochondrial (E. coli); metastasis associated lung adenocarcinoma transcript 1 (non-protein coding); aurora kinase A interacting protein 1; ubiquitin conjugating enzyme E2H; AF4/FMR2 family, member 4; QKI, KH domain containing, RNA binding; regulator of calcineurin 1; abl-interactor 2; p21 protein (Cdc42/Rac)-activated kinase 2; RAB11B, member RAS oncogene family; MYC associated factor X; sprouty-related, EVH1 domain containing 2; eukaryotic translation initiation factor 4 gamma, 3; ring finger protein (C3H2C3 type) 6; ribosomal protein, large, P2; v-akt murine thymoma viral oncogene homolog 2; zinc finger, DHHC-type containing 20; histone cluster 1, H1e; hyaluronan synthase 3; amyloid beta (A4) precursor-like protein 2; oxysterol binding protein-like 8; heterogeneous nuclear ribonucleoprotein H1 (H); OTU deubiquitinase 4; septin 11; CD44 molecule (Indian blood group); cytoskeleton-associated protein 4; protein phosphatase, Mg2+/Mn2+ dependent, 1K; v-akt murine thymoma viral oncogene homolog 3; neurofibromin 2 (merlin); ATPase, H+ transporting, lysosomal accessory protein 2; lysine (K)-specific demethylase 4B; SLAIN motif family member 2; transforming growth factor beta 2; zinc finger protein 460; twinfilin actin binding protein 1; synaptosome associated protein 23 kDa; phosphoprotein enriched in astrocytes 15; glucosaminyl (N-acetyl) transferase 4, core 2; AF4/FMR2 family, member 1; RNA polymerase II associated protein 3; proline rich 14; collagen, type VI, alpha 1; protein tyrosine phosphatase, non-receptor type 11; phosphodiesterase 1C, calmodulin-dependent 70 kDa; zinc finger, CCHC domain containing 7; PDZ and LIM domain 4; 5-azacytidine induced 2; heat shock protein, alpha-crystallin-related, B6; Ras homolog enriched in brain pseudogene; ribosomal protein L27; dystroglycan 1 (dystrophin-associated glycoprotein 1); protein kinase N2; caveolin 2; DEAH (Asp-Glu-Ala-His) box helicase 9; uncharacterized LOC105374954; tyrosine 3-monooxygenase/tryptophan 5-monooxygenase activation protein, epsilon; NCK-associated protein 1; transmembrane protein 259; solute carrier family 7 (cationic amino acid transporter, y+ system), member 1; sarcoglycan beta; B double prime 1, subunit of RNA polymerase III transcription initiation factor IIIB; lysine (K)-specific methyltransferase 2C; aspirin; solute carrier family 6 (neurotransmitter transporter), member 6; IK cytokine, down-regulator of HLA II; transmembrane and coiled-coil domains 6; TROVE domain family, member 2; tumor necrosis factor receptor superfamily, member 11b; centrosomal protein 89 kDa; protein tyrosine phosphatase, non-receptor type 12; dynein, cytoplasmic 1, heavy chain 1; annexin A2; chromobox homolog 5; CDV3 homolog (mouse); mitochondrial ribosomal protein L52; GATA zinc finger domain containing 2A; RAB7A, member RAS oncogene family; TRAF-type zinc finger domain containing 1; transforming growth factor beta 2; H3 histone, family 3A; protein phosphatase 4, regulatory subunit 3A; protein tyrosine phosphatase, non-receptor type 12; ribosomal protein L5; small nucleolar RNA, H/ACA box 66; transmembrane protein 259; iduronate 2-sulfatase; ATPase, class VI, type 11B; fibroblast growth factor receptor substrate 2; transcription factor AP-2 alpha (activating enhancer binding protein 2 alpha); RNA binding protein, fox-1 homolog (C. elegans) 2; acidic nuclear phosphoprotein 32 family member A; mitochondrial ribosomal protein L41; interleukin 6 signal transducer; elaC ribonuclease Z 2; uncharacterized LOC101926943; transmembrane protein 33; signal sequence receptor, gamma (translocon-associated protein gamma); chloride intracellular channel 4; radixin; calreticulin; HECT domain containing E3 ubiquitin protein ligase 1; transmembrane protein 223; large tumor suppressor kinase 1; endosulfine alpha; homeodomain interacting protein kinase 2; protein kinase C, iota; transducin-like enhancer of split 4; zinc finger CCCH-type, antiviral 1-like; schlafen family member 5; zinc finger, AN1-type domain 5; sperm specific antigen 2; DEAD (Asp-Glu-Ala-Asp) box helicase 17; dynein, cytoplasmic 1, light intermediate chain 2; neuron navigator 3; dephospho-CoA kinase domain containing; protein tyrosine phosphatase type IVA, member 1; methionine adenosyltransferase II, beta; protein kinase, cAMP-dependent, catalytic, alpha; heterogeneous nuclear ribonucleoprotein U-like 1; ras homolog family member A; TBC1 domain family, member 2B; pleckstrin homology-like domain, family A, member 2; vacuolar protein sorting 53 homolog (S. cerevisiae); Cbp/p300-interacting transactivator, with Glu/Asp rich carboxy-terminal domain, 2; protein phosphatase 1, regulatory (inhibitor) subunit 14A; gamma-glutamyl carboxylase; profilin 1; minichromosome maintenance 9 homologous recombination repair factor; solute carrier family 39, member 9; protein associated with topoisomerase II homolog 1 (yeast); protein phosphatase 1, regulatory (inhibitor) subunit 2; catenin (cadherin-associated protein), beta 1; Nipped-B homolog (Drosophila); LSM12 homolog; protein phosphatase, Mg2+/Mn2+ dependent, 1K; microtubule associated protein 4; TAF9B RNA polymerase II, TATA box binding protein (TBP)-associated factor, 31 kDa; teneurin transmembrane protein 3; Myb/SANT-like DNA-binding domain containing 4 with coiled-coils; biliverdin reductase A; WWTR1 antisense RNA 1; Yip1 domain family member 5; phosphoglycerate kinase 1; serine/threonine/tyrosine interacting protein; transmembrane protein 165; chromosome 5 open reading frame 15; ST3 beta-galactoside alpha-2,3-sialyltransferase 6; glucosamine (N-acetyl)-6-sulfatase; discs, large homolog 1 (Drosophila); TPT1 antisense RNA 1; hematological and neurological expressed 1; phosphatidylinositol-4-phosphate 3-kinase, catalytic subunit type 2 alpha; exocyst complex component 5; nuclear paraspeckle assembly transcript 1 (non-protein coding); histone cluster 1, H2ae; GTPase activating protein (SH3 domain) binding protein 2; vesicle associated membrane protein 5; EH domain containing 2; neuron navigator 3; uncharacterized LOC100506403; runt-related transcription factor 1; SPT6 homolog, histone chaperone; desumoylating isopeptidase 2; histone cluster 2, H2aa3; histone cluster 2, H2aa4; AT rich interactive domain 5B (MRF1-like); TRAF family member-associated NFKB activator; DEAD (Asp-Glu-Ala-Asp) box helicase 3, X-linked; ecotropic viral integration site 5; F-box protein 32; exosome component 3; neudesin neurotrophic factor; mitochondrial ribosomal protein S12; translocated promoter region, nuclear basket protein; uncharacterized LOC100506403; runt-related transcription factor 1; Rho GTPase activating protein 1; pancreatic progenitor cell differentiation and proliferation factor; SKI-like proto-oncogene; ARP2 actin-related protein 2 homolog (yeast); CD46 molecule, complement regulatory protein; ATPase type 13A3; voltage-dependent anion channel 3; activating transcription factor 6 beta; tenascin XB; tripeptidyl peptidase II; limb and CNS expressed 1 like; calnexin; MYC-associated zinc finger protein (purine-binding transcription factor); zinc finger, MYM-type 5; endothelial PAS domain protein 1; lysyl oxidase-like 2; fumarate hydratase; 6-phosphogluconolactonase; trimethylguanosine synthase 1; glutamyl-prolyl-tRNA synthetase; VMA21 vacuolar H+-ATPase homolog (S. cerevisiae); ADAMTS like 4; A kinase (PRKA) anchor protein 13; myeloid cell leukemia 1; RAB35, member RAS oncogene family; CTD small phosphatase like 2; guanylate kinase 1; nuclear receptor subfamily 2, group C, member 2; protein phosphatase 1, regulatory (inhibitor) subunit 11; prostaglandin 12 (prostacyclin) receptor (IP); DEAD (Asp-Glu-Ala-Asp) box helicase 42; secretory carrier membrane protein 1; prostaglandin E receptor 3 (subtype EP3); 3-phosphoinositide dependent protein kinase 1; AF4/FMR2 family, member 4; PRKC, apoptosis, WT1, regulator; plexin A3; neudesin neurotrophic factor; discoidin domain receptor tyrosine kinase 2; KIAA0430; chromosome 16 open reading frame 72; DEAD (Asp-Glu-Ala-Asp) box helicase 3, Y-linked; general transcription factor IIA 1; transmembrane 9 superfamily member 3; sorting nexin 19; FCH domain only 2; TOX high mobility group box family member 4; NOP16 nucleolar protein; hook microtubule-tethering protein 3; zinc finger protein 460; AP2 associated kinase 1; suppressor of IKBKE 1; RPA interacting protein; zinc finger protein, X-linked, zinc finger protein, Y-linked; guanine nucleotide binding protein (G protein), q polypeptide; Rho GTPase activating protein 35; spectrin, beta, non-erythrocytic 1; FCF1 rRNA-processing protein; matrix metallopeptidase 24 (membrane-inserted); tyrosine 3-monooxygenase/tryptophan 5-monooxygenase activation protein, zeta; retinoic acid receptor, beta; catenin (cadherin-associated protein), alpha 1; phosphatidylinositol-4-phosphate 5-kinase, type 1, alpha; leucine rich repeat containing 28; bromodomain adjacent to zinc finger domain 1B; coiled-coil domain containing 186; phosphatidylinositol-4-phosphate 5-kinase, type 1, alpha; density-regulated protein; BUB3 mitotic checkpoint protein; inositol monophosphatase domain containing 1; La ribonucleoprotein domain family, member 4B; poly(A) polymerase alpha; Sp3 transcription factor; activating transcription factor 1; short coiled-coil protein; serine/arginine-rich splicing factor 10; clathrin, light chain B; tensin 1; metastasis associated lung adenocarcinoma transcript 1 (non-protein coding); neutral sphingomyelinase activation associated factor; sarcoglycan delta; early endosome antigen 1; RAN binding protein 1; calmodulin 1 (phosphorylase kinase, delta), calmodulin 2 (phosphorylase kinase, delta); programmed cell death 1 ligand 2; tousled-like kinase 1; structural maintenance of chromosomes 1A; transforming growth factor beta receptor II; histamine N-methyltransferase; LYR motif containing 2; phosphatidylinositol-4-phosphate 3-kinase, catalytic subunit type 2 alpha; casein kinase 1, alpha 1; CUB domain containing protein 1; stathmin 2; SP110 nuclear body protein; thioredoxin-related transmembrane protein 1; protease, serine, 23; PDZ and LIM domain 5; tetratricopeptide repeat domain 37; homeodomain interacting protein kinase 3; centrosomal protein 89 kDa; zinc finger, BED-type containing 6; inhibin beta A; mitogen-activated protein kinase kinase kinase 2; transcription factor binding to IGHM enhancer 3; dickkopf WNT signaling pathway inhibitor 3; fibroblast growth factor 14; histone cluster 1, H2bf; heat shock protein 90 kDa alpha (cytosolic), class B member 1; ras homolog family member B; U2AF homology motif (UHM) kinase 1; calumenin; akirin 2; transcription factor binding to IGHM enhancer 3; transmembrane and tetratricopeptide repeat containing 3; related RAS viral (r-ras) oncogene homolog 2; ubiquitin specific peptidase 34; growth arrest and DNA-damage-inducible, beta; vesicle associated membrane protein 2; solute carrier family 12 (potassium/chloride transporter), member 6; retinol dehydrogenase 10 (all-trans); SLAIN motif family member 2; phosphatidylinositol binding clathrin assembly protein; solute carrier family 8 (sodium/calcium exchanger), member 1; coiled-coil domain containing 88A; VPS35 retromer complex component; arginyl-tRNA synthetase 2, mitochondrial; SET domain containing 2; protein tyrosine phosphatase, receptor type, A; polymerase (RNA) II (DNA directed) polypeptide A, 220 kDa; CD46 molecule, complement regulatory protein; zinc finger protein 460; lysine (K)-specific methyltransferase 2D; casein kinase 1, delta; AT rich interactive domain 1A (SWI-like); TIMP metallopeptidase inhibitor 3; ceramide synthase 6; maternally expressed 3 (non-protein coding); AHNAK nucleoprotein 2; succinate dehydrogenase complex, subunit C, integral membrane protein, 15 kDa; transmembrane and coiled-coil domains 3; RAB6A, member RAS oncogene family; DEAD (Asp-Glu-Ala-Asp) box helicase 3, X-linked; YTH domain containing 1; S-phase kinase-associated protein 2, E3 ubiquitin protein ligase; zinc finger RNA binding protein; wingless-type MMTV integration site family, member 5B; selenoprotein T; CDK2-associated, cullin domain 1; heterogeneous nuclear ribonucleoprotein D like; TROVE domain family, member 2; Y box binding protein 3; ring finger protein 141; uncharacterized LOC100506403, runt-related transcription factor 1; jumonji domain containing 1C; nuclear factor I/X (CCAAT-binding transcription factor); ACTA2 antisense RNA 1; Ras association (RaIGDS/AF-6) domain family member 3; TSC22D1 antisense RNA 1; microtubule associated protein 1A; spastic paraplegia 20 (Troyer syndrome); serine/threonine kinase 3; SLC2A4 regulator; family with sequence similarity 171, member B; fibroblast growth factor receptor 2; histone cluster 1, H2bh; centrosomal protein 89 kDa; AF4/FMR2 family, member 4; cortactin; transducin-like enhancer of split 4; family with sequence similarity 171, member B; RAB5C, member RAS oncogene family; NADH dehydrogenase, subunit 6 (complex I); tyrosylprotein sulfotransferase 2; G1 to S phase transition 1; UDP-glucose glycoprotein glucosyltransferase 2; collagen, type XII, alpha 1; establishment of sister chromatid cohesion N-acetyltransferase 1; cleavage and polyadenylation specific factor 2; septin 9; DnaJ (Hsp40) homolog, subfamily B, member 5; ubiquitin-like modifier activating enzyme 6; DnaJ (Hsp40) homolog, subfamily C, member 13; protein tyrosine phosphatase, receptor type, G; caldesmon 1; myosin VA; NADH dehydrogenase (ubiquinone) Fe-S protein 1, 75 kDa (NADH-coenzyme Q reductase); zinc finger protein 146; ataxin 7-like 3B; mannosidase, alpha, class 1A, member 2; U2AF homology motif (UHM) kinase 1; SOS Ras/Rho guanine nucleotide exchange factor 2; mitogen-activated protein kinase kinase kinase kinase 5; desumoylating isopeptidase 2; calumenin; proline-rich coiled-coil 2C; sidekick cell adhesion molecule 2; stress-induced phosphoprotein 1; adenomatous polyposis coli; calcium/calmodulin-dependent protein kinase kinase 2, beta; cerebral endothelial cell adhesion molecule; nuclear factor of activated T-cells 5, tonicity-responsive; tetratricopeptide repeat domain 3; RNA binding motif protein 8A; BCL2-like 1; pre-B-cell leukemia homeobox 2; hyaluronan synthase 3, UTP4 small subunit (SSU) processome component; heat shock protein 90 kDa beta (Grp94), member 1; RAN binding protein 9; neuregulin 1; dual specificity phosphatase 1; TSR3, 20S rRNA accumulation, homolog (S. cerevisiae); NSL1, MIS12 kinetochore complex component; histone cluster 1, H2be; homeodomain interacting protein kinase 1; DET1 and DDB1 associated 1; protein kinase, DNA-activated, catalytic polypeptide; tripartite motif containing 8; N(alpha)-acetyltransferase 30, NatC catalytic subunit; AT rich interactive domain 4B (RBP1-like), RNA binding motif protein 34; synaptopodin 2; platelet-activating factor acetylhydrolase 1b, catalytic subunit 2 (30 kDa); uncharacterized LOC101243545; ELK4, ETS-domain protein (SRF accessory protein 1); chromosome 5 open reading frame 22; SAM domain and HD domain 1; crystallin zeta like 1; PHD finger protein 20; ubiquitin specific peptidase 12; PDZ and LIM domain 7 (enigma); TAF9B RNA polymerase II, TATA box binding protein (TBP)-associated factor, 31 kDa; pleckstrin homology-like domain, family B, member 2; ADAM metallopeptidase domain 9; T cell activation inhibitor, mitochondrial; MIS18 binding protein 1; YKT6 v-SNARE homolog (S. cerevisiae); ring finger protein 207; schlafen family member 5; paired related homeobox 1; syntrophin, beta 2 (dystrophin-associated protein A1, 59 kDa, basic component 2); torsin A interacting protein 2; phosphoinositide-3-kinase, regulatory subunit 2 (beta); DCN1, defective in cullin neddylation 1, domain containing 1; LRRC75A antisense RNA 1; cytoplasmic polyadenylation element binding protein 2; nuclear receptor subfamily 2, group F, member 2; ribosomal protein L38; HIV-1 Tat specific factor 1; RAB12, member RAS oncogene family; ezrin; GM2 ganglioside activator; pericentriolar material 1; suppressor of cytokine signaling 4; calcium/calmodulin-dependent serine protein kinase (MAGUK family); heat shock transcription factor 2; nuclear casein kinase and cyclin-dependent kinase substrate 1; transformer 2 beta homolog (Drosophila); H2B histone family, member S (pseudogene), histone cluster 1, H2bk, histone H2B type F-S-like; lysine (K)-specific demethylase 1B; Ras association (RaIGDS/AF-6) domain family (N-terminal) member 8; R-spondin 4; programmed cell death 5; itchy E3 ubiquitin protein ligase; regulation of nuclear pre-mRNA domain containing 1A; zinc finger protein 770; galactokinase 1; protein phosphatase 3, catalytic subunit, gamma isozyme; solute carrier family 30 (zinc transporter), member 4; MRS2 magnesium transporter; KxDL motif containing 1; secretion associated, Ras related GTPase 1B; bromodomain adjacent to zinc finger domain 1B; nestin; FK506 binding protein 1A; lectin, mannose-binding, 1; synaptopodin 2; laminin, alpha 4; twinfilin actin binding protein 1, twinfilin 1 pseudogene 1; ankyrin 2, neuronal; cyclin-dependent kinase 6; human immunodeficiency virus type I enhancer binding protein 2; zinc finger, MYND-type containing 11; metastasis suppressor 1-like; cullin 4B; polymerase (DNA directed), eta; signal transducing adaptor molecule (SH3 domain and ITAM motif) 2; heterogeneous nuclear ribonucleoprotein U (scaffold attachment factor A); serine/threonine kinase 4; insulin like growth factor binding protein 2; NOP16 nucleolar protein; peptidyl arginine deiminase, type II; guanine nucleotide binding protein (G protein), gamma 12; small nucleolar RNA, C/D box 3A, small nucleolar RNA, C/D box 3B-1, small nucleolar RNA, C/D box 3B-2, small nucleolar RNA, C/D box 3C; Shwachman-Bodian-Diamond syndrome, Shwachman-Bodian-Diamond syndrome pseudogene 1; Josephin domain containing 2; WD repeat domain 1; ubiquitin protein ligase E3C; coiled-coil domain containing 174; maternally expressed 3 (non-protein coding); chromodomain helicase DNA binding protein 9; abhydrolase domain containing 2; leucine rich repeat (in FLII) interacting protein 1; MET proto-oncogene, receptor tyrosine kinase; neurofibromin 1; v-akt murine thymoma viral oncogene homolog 3; lethal giant larvae homolog 1 (Drosophila); gap junction protein gamma 1; Cbp/p300-interacting transactivator, with Glu/Asp rich carboxy-terminal domain, 2; SH3 and PX domains 2A; tropomyosin 4; homeodomain interacting protein kinase 3; ankyrin repeat domain 19, pseudogene; aldehyde dehydrogenase 1 family, member L2; junctional adhesion molecule 3; interleukin 1 receptor, type I; cysteine rich hydrophobic domain 2; family with sequence similarity 168, member B; angiomotin like 1; EH domain binding protein 1; GM2 ganglioside activator; tyrosine 3-monooxygenase/tryptophan 5-monooxygenase activation protein, eta; erythrocyte membrane protein band 4.1-like 1; heterogeneous nuclear ribonucleoprotein U (scaffold attachment factor A); peroxisomal biogenesis factor 13; neurofibromin 1; SH3 domain binding glutamate-rich protein like 3; FGFR1OP N-terminal like; G-rich RNA sequence binding factor 1; guanine nucleotide binding protein (G protein), alpha 13; ATPase, Ca++ transporting, cardiac muscle, slow twitch 2; collagen, type I, alpha 1; alpha thalassemia/mental retardation syndrome X-linked; nuclear factor I/X (CCAAT-binding transcription factor); nuclear factor I/C (CCAAT-binding transcription factor); stromal antigen 3-like 1 (pseudogene), stromal antigen 3-like 2 (pseudogene), stromal antigen 3-like 3 (pseudogene); mannosidase, endo-alpha; keratin 17, type I; atrophin 1; RAB30, member RAS oncogene family; crystallin beta B2, crystallin beta B2 pseudogene 1; PHD finger protein 12; uncharacterized LOC100190986; KDEL (Lys-Asp-Glu-Leu) endoplasmic reticulum protein retention receptor 1; RaI GEF with PH domain and SH3 binding motif 2; HECT, UBA and WWE domain containing 1, E3 ubiquitin protein ligase; prickle homolog 3; LIM domain containing preferred translocation partner in lipoma; TMF1-regulated nuclear protein 1; purine-rich element binding protein B; NADH dehydrogenase (ubiquinone) Fe-S protein 8, 23 kDa (NADH-coenzyme Q reductase); laccase (multicopper oxidoreductase) domain containing 1; syntaxin 6; zinc finger, MYND-type containing 11; spectrin, beta, non-erythrocytic 1; BRICK1, SCAR/WAVE actin-nucleating complex subunit; protein associated with topoisomerase II homolog 1 (yeast); RAR-related orphan receptor A; discs, large (Drosophila) homolog-associated protein 4; F-box and leucine-rich repeat protein 20; solute carrier family 25 (aspartate/glutamate carrier), member 13; carboxypeptidase D; AF4/FMR2 family, member 4; neuroblastoma amplified sequence; cytochrome c oxidase subunit VIIc; G elongation factor, mitochondrial 2; ribulose-5-phosphate-3-epimerase, ribulose-5-phosphate-3-epimerase-like 1; ArfGAP with GTPase domain, ankyrin repeat and PH domain 2; ubiquitin specific peptidase 10; dedicator of cytokinesis 9; kinesin family member 1B; lysine (K)-specific methyltransferase 2D; ATP binding cassette subfamily D member 3; ADP-ribosylation factor interacting protein 1; Sp1 transcription factor; biorientation of chromosomes in cell division 1-like 1; ArfGAP with FG repeats 1; general transcription factor lii; eukaryotic translation initiation factor 4E family member 3; CASP8 and FADD like apoptosis regulator; leucyl-tRNA synthetase; secretion associated, Ras related GTPase 1B; cullin 5; polymerase (RNA) II (DNA directed) polypeptide J4, pseudogene; WNT1 inducible signaling pathway protein 3; RAB3B, member RAS oncogene family; gamma-aminobutyric acid (GABA) B receptor, 2; IWS1 homolog (S. cerevisiae); interleukin 13 receptor, alpha 1; PDZ and LIM domain 7 (enigma); vascular endothelial growth factor A; primase, DNA, polypeptide 2 (58 kDa); cardiolipin synthase 1; zinc finger protein 655; mex-3 RNA binding family member C; gap junction protein alpha 5; casein kinase 1, alpha 1; interactor of little elongation complex ELL subunit 2; zinc finger protein 326; Ellis van Creveld protein; integrin beta 1; mohawk homeobox; required for meiotic nuclear division 5 homolog B; DEAD (Asp-Glu-Ala-Asp) box helicase 17; v-akt murine thymoma viral oncogene homolog 2; sperm associated antigen 9; factor interacting with PAPOLA and CPSF1; histone cluster 1, H2aj; heat shock transcription factor 1; leukocyte receptor cluster (LRC) member 8; MDM2 proto-oncogene, E3 ubiquitin protein ligase; mitochondrial ribosomal protein L30; tubulin, beta 2A class IIa; tubulin, beta 2B class IIb; golgi-associated PDZ and coiled-coil motif containing; DNA replication and sister chromatid cohesion 1; forkhead box O3, forkhead box O3B pseudogene; sprouty-related, EVH1 domain containing 1; RaI GEF with PH domain and SH3 binding motif 2; LSM family member 14B; dermatan sulfate epimerase-like; retinoblastoma binding protein 9; mitogen-activated protein kinase kinase kinase 7; methylenetetrahydrofolate dehydrogenase (NADP+ dependent) 2, methenyltetrahydrofolate cyclohydrolase; SMG7 nonsense mediated mRNA decay factor; FK506 binding protein 15; argonaute RISC catalytic component 2; heterogeneous nuclear ribonucleoprotein U-like 2; sarcolemma associated protein; DEAD (Asp-Glu-Ala-Asp) box helicase 17; ribosomal protein S6 kinase, 70 kDa, polypeptide 1; NADH dehydrogenase (ubiquinone) 1 beta subcomplex, 7, 18 kDa; MLX interacting protein; GABPB1 antisense RNA 1; SWI/SNF related, matrix associated, actin dependent regulator of chromatin, subfamily a, member 1; zinc finger, MYM-type 6; zinc finger and BTB domain containing 20; capping protein (actin filament) muscle Z-line, beta; Kruppel-like factor 12; sestrin 3; MOB kinase activator 1A; KRAB box domain containing 4, zinc finger protein 674; phosphatidylinositol-4,5-bisphosphate 3-kinase, catalytic subunit beta; N-ethylmaleimide-sensitive factor attachment protein, gamma; mitogen-activated protein kinase kinase kinase 7; MORN repeat containing 2; ras homolog family member J; TRPM8 channel-associated factor 1; cAMP-regulated phosphoprotein 19 kDa; 5′-nucleotidase, ecto (CD73); transducin (beta)-like 1 X-linked receptor 1; spectrin, beta, non-erythrocytic 1; importin 8; histone cluster 1, H2bi; cofilin 1 (non-muscle); phosphate cytidylyltransferase 1, choline, alpha; lysine (K)-specific methyltransferase 2A; palmdelphin; zinc finger and BTB domain containing 24; tropomodulin 3 (ubiquitous); sushi, von Willebrand factor type A, EGF and pentraxin domain containing 1; ubiquitin conjugating enzyme E2G 2; acid phosphatase 1, soluble; cell division cycle 27; xylosyltransferase I; gamma-aminobutyric acid (GABA) B receptor, 2; GATA zinc finger domain containing 2B; iduronate 2-sulfatase; integrator complex subunit 1; ubiquitination factor E4B; ankyrin repeat and FYVE domain containing 1; high mobility group box 1; TAF9B RNA polymerase II, TATA box binding protein (TBP)-associated factor, 31 kDa; collagen, type VI, alpha 1; ubiquitin specific peptidase 25; sestrin 3; F-box and WD repeat domain containing 11; histone cluster 1, H2ai; synaptosome associated protein 23 kDa; pre-mRNA processing factor 18; spermatogenesis associated 5; huntingtin interacting protein K, small EDRK-rich factor 2; SERF2-C15orf63 readthrough; myeloid/lymphoid or mixed-lineage leukemia, translocated to, 1; spectrin, beta, non-erythrocytic 1; solute carrier family 3 (amino acid transporter heavy chain), member 1; signal recognition particle 72 kDa; ubiquitin specific peptidase 32; sorbin and SH3 domain containing 3; thioredoxin interacting protein; HOXD antisense growth-associated long noncoding RNA; uncharacterized LOC100506403, runt-related transcription factor 1; ring finger protein 168, E3 ubiquitin protein ligase; RAD21 cohesin complex component; family with sequence similarity 198, member B; ubiquitin specific peptidase 33; PR domain containing 2, with ZNF domain; polycomb group ring finger 5; GLI family zinc finger 4; integrin alpha 6; cytoplasmic linker associated protein 2; guanine nucleotide binding protein (G protein), beta polypeptide 4; glutaminase; centrosomal protein 63 kDa; voltage-dependent anion channel 1; transcription factor Dp-1; chromosome 6 open reading frame 106; lactamase, beta; HERPUD family member 2; large tumor suppressor kinase 1; ezrin; SUMO1/sentrin specific peptidase 5; chaperonin containing TCP1, subunit 2 (beta); zinc finger protein 780A, zinc finger protein 780B; coatomer protein complex subunit gamma 1; RAB23, member RAS oncogene family; tripartite motif containing 8; RB-associated KRAB zinc finger; sema domain, immunoglobulin domain (Ig), short basic domain, secreted, (semaphorin) 3D; polymerase I and transcript release factor; retinoblastoma binding protein 6; heterogeneous nuclear ribonucleoprotein H3 (2H9); mitochondrial ribosomal protein L19; sortilin 1; protocadherin gamma subfamily A, 11; polypyrimidine tract binding protein 1; exosome component 3; dual specificity phosphatase 3; cyclin-dependent kinase 13; paralemmin 2, PALM2-AKAP2 readthrough; PX domain containing 1; Rho guanine nucleotide exchange factor (GEF) 12; mitochondrial ribosomal protein L57; histone cluster 1, H2bd; squalene epoxidase; ubiquitin conjugating enzyme E2H; ubiquitin specific peptidase 22; growth differentiation factor 15; prostaglandin E receptor 3 (subtype EP3); glycosyltransferase like domain containing 1; GABA(A) receptor-associated protein like 1; phosphatidylinositol glycan anchor biosynthesis class X; CDC42 small effector 1; basic helixloop-helix family, member e41; TOR signaling pathway regulator; CCR4-NOT transcription complex subunit 4; sorting nexin 25; ring finger and CCCH-type domains 1; tankyrase, TRF1-interacting ankyrin-related ADP-ribose polymerase; coiled-coil domain containing 58; plectin; golgin A8 family, member A; THUMP domain containing 3; mannosidase, endo-alpha; cytohesin 3; chaperonin containing TCP1, subunit 4 (delta); and ring finger and CCCH-type domains 2.

Genes expressed more in androgen non-inhibited cells compared with androgen-inhibited cells and which are also found in both intact DP cells (obtained from follicles obtained in Example 2, and isolated as described in Example 1) and cultured DP cells (obtained as described in Example 1) are shown below (List C):

spalt-like transcription factor 2; interferon regulatory factor 1; PSMDS antisense RNA 1 (head to head); CTTNBP2 N-terminal like; neutral sphingomyelinase activation associated factor; guanylate cyclase 1, soluble, beta 3; trichoplein, keratin filament binding; leucine rich repeat containing 17; nucleolar protein 11; glutathione S-transferase mu 5; protein arginine methyltransferase 3; DEAD (Asp-Glu-Ala-Asp) box polypeptide 23; methyltransferase like 13; alcohol dehydrogenase 5 (class III), chi polypeptide; erythrocyte membrane protein band 4.1 like 5; glutathione peroxidase 3; chromatin accessibility complex 1; pumilio RNA binding family member 3; transcriptional adaptor 2B; ubiquitin-fold modifier 1; SP140 nuclear body protein-like; ribosomal RNA processing 36; BEN domain containing 3; derlin 2; ArfGAP with coiled-coil, ankyrin repeat and PH domains 3; major histocompatibility complex, class I, E; growth arrest-specific 7; transporter 2, ATP-binding cassette, sub-family B (MDR/TAP); MHC class I polypeptide-related sequence B; ribosomal protein L31; SERTA domain containing 4; XAGE-4 protein; phosphofurin acidic cluster sorting protein 2; coiled-coil domain containing 51; LUC7-like 3 pre-mRNA splicing factor; pericentriolar material 1; ubiquitin-conjugating enzyme E2D 4 (putative); forkhead box P2; formin binding protein 4; and ubiquinol-cytochrome c reductase binding protein.

Genes expressed more in androgen-inhibited cells compared with androgen non-inhibited cells and which are also expressed in both intact DP cells and cultured DP cells are shown below (List D):

tachykinin 4 (hemokinin); zinc finger protein 816; glial cell derived neurotrophic factor; solute carrier family 5 (sodium/glucose cotransporter), member 2; TP53 target 1 (non-protein coding); ARPC4-TTLL3 readthrough, tubulin tyrosine ligase-like family member 3; discoidin, CUB and LCCL domain containing 2; diacylglycerol lipase, beta; breast cancer estrogen-induced apoptosis 2; serum amyloid A1, serum amyloid A2; SAA2-SAA4 readthrough; uncharacterized LOC101929450; transcription factor 3; RNA binding motif protein 33; PRKC, apoptosis, WT1, regulator; v-maf avian musculoaponeurotic fibrosarcoma oncogene homolog; syndecan 1; neuron navigator 1; peroxisomal biogenesis factor 14; Fanconi anemia core complex associated protein 24; zinc finger protein 528; Sin3A associated protein 18 kDa; casein kinase 1, gamma 1; uncharacterized LOC648987; centrosomal protein 290 kDa; exocyst complex component 3-like 1; crystallin alpha B; autophagy related 4D, cysteine peptidase; proteasome 26S subunit, non-ATPase 8; protein tyrosine phosphatase, non-receptor type 23; MKL/myocardin-like 2; spastic paraplegia 11 (autosomal recessive); DENN/MADD domain containing 5B; zinc finger protein 205; SER-TAD4 antisense RNA 1; phospholipid phosphatase related 3; WW domain binding protein 11; iduronidase, alpha-L-; salvador family WW domain containing protein 1; leukosialin; sialophorin; C-type lectin domain family 7, member A; ras-related C3 botulinum toxin substrate 1 (rho family, small GTP binding protein Rac1); SLC2A4 regulator; THAP domain containing, apoptosis associated protein 2; leukosialin, sialophorin; nuclear transcription factor Y subunit gamma; latent transforming growth factor beta binding protein 4; ubiquitin protein ligase E3 component n-recognin 3 (putative); uncharacterized LOC105375666; centrosomal protein 104 kDa; zinc finger protein 626; zinc finger homeobox 3; ilvB (bacterial acetolactate synthase)-like; zinc finger and BTB domain containing 7A; trimethylguanosine synthase 1; atlastin GTPase 3; twisted gastrulation BMP signaling modulator 1; activating transcription factor 5; nuclear factor, erythroid 2-like 2; C2 calcium-dependent domain containing 4B; transforming growth factor beta regulator 1; elastin; dynactin 1; 1acylglycerol-3-phosphate O-acyltransferase 3; chloride channel CLIC-like 1; ArfGAP with coiled-coil, ankyrin repeat and PH domains 2; transmembrane protein 259; RNA binding motif protein 14; fucosidase, alpha-L-1, tissue; chromatin target of PRMT1; ELF1 homolog, elongation factor 1; TRPM8 channel-associated factor 1; NHL repeat containing 2; integrin beta 1, integrin beta 1 pseudogene 1; tetratricopeptide repeat domain 3; inverted formin, FH2 and WH2 domain containing; trinucleotide repeat containing 6A; methylthioadenosine phosphorylase; myosin, heavy chain 11, smooth muscle; ataxin 1; calcium/calmodulin-dependent protein kinase II inhibitor 1; GTP binding protein 2; ubiquitin protein ligase E3B; early growth response 1; actin related protein 2/3 complex subunit 5; chaperonin containing TCP1, subunit 4 (delta); Rho guanine nucleotide exchange factor (GEF) 12; PX domain containing 1; cytoplasmic linker associated protein 2; pre-mRNA processing factor 18; GABPB1 antisense RNA 1; LSM family member 14B; KxDL motif containing 1; R-spondin 4; H2B histone family, member S (pseudogene), histone cluster 1, H2bk, histone H2B type F-S-like; NSL1, MIS12 kinetochore complex component; heat shock protein 90 kDa beta (Grp94), member 1; TSC22D1 antisense RNA 1; transcription factor binding to IGHM enhancer 3; Sp3 transcription factor; myeloid cell leukemia 1; TBC1 domain family, member 2B; zinc finger, AN1-type domain 5; protein tyrosine phosphatase, non-receptor type 12; H3 histone, family 3A; GATA zinc finger domain containing 2A; IK cytokine, down-regulator of HLA II, transmembrane and coiled-coil domains 6; protein kinase N2; ets variant 5; and collagen, type VI, alpha 1.

Selections of one or two or more genes from Lists C and D may be used in quality control (QC) tests on cultured cells to confirm whether or not they are androgen non-inhibited. The genes in List C may be particularly useful as they could differentiate between both androgen-inhibited and androgen non-inhibited cells, for example either in intact DPs before amplification in culture or after the cells have been cultured. Differentiating genes that express cell surface proteins could also be used as antigens for a variety of cell separation techniques.

For genes above from Lists A-D, unique Affymetrix ID numbers and gene symbols are available in the Affymetrix U133 Plus 2.0 Array kit.

### Example 5

### Identification and Selection of Androgen Non-Inhibited Cells

At the end of the amplification cells and before clinical use on the patient, cells are assessed for the expression of biomarkers as described in Example 4. In particular, using the genes set out in Table 1 and/or List A-D, it is possible to discriminate between androgen non-inhibited, hair follicle inductive cells and androgen-sensitive cells. Expression or non-expression of appropriate control genes known in the art can be tested for comparative purposes.

The desired androgen non-inhibited cells are either positively or negatively selected, or both. The former approach aims at isolating the androgen non-inhibited cell type from the entire population, while the latter strategy involves the depletion of androgen inhibited cells from the population resulting in only androgen non-inhibited cells remaining. Specific binding of surface antigens to either antibodies or aptamers can selectively capture cells where differentially expressed genes (for example, as disclosed in Example 4) encode cell surface proteins (e.g. integrins). The captured cells are subsequently detected with the help of measurable probes—for example fluorochromes or magnetic particles—with which the antibodies/aptamers are labeled. Once labelled, the cells are selected either positively or negatively using techniques such as fluorescent activated cell sorting FACS or magnetically activated cell sorting (MACS).

### Example 6

### Preparation of Cells Prior to Injection

At the end of the culture, cells are prepared for injection. Cells are harvested by trypsinising from the dishes, collected by centrifugation and re-suspended in Hypothermosol-FRS (Biolife).

### Example 7

### Injection of Cells

Androgen non-inhibited cells amplified in vitro are counted and injected close to a miniaturised hair located in a balding or pre-balding region of the scalp using a micro syringe with a 27-G needle. (Cells can be injected through a needle as small as 30-G, but 27-G is preferred).

1. A method for rejuvenating hair follicles, the method comprising the steps of:
(1) obtaining androgen non-inhibited cells from hair follicle tissue;
(2) culturing the androgen non-inhibited cells obtained in step (1) under conditions suitable for proliferation, to produce an expanded population of androgen-non-inhibited cells; and
(3) implanting the expanded population of androgen non-inhibited cells produced in step (2) proximal to miniaturised and/or miniaturising hair follicles, thereby rejuvenating the miniaturised and/or miniaturising hair follicles.

2. The method according to claim 1, wherein step (1) and/or (2) includes a step of selecting and/or androgen non-inhibited cells from a mixed population of cells from hair follicle tissue.

3. The method of either claim 1 or 2, wherein the androgen non-inhibited cells are androgen insensitive cells, for example wherein the hair follicle tissue is scalp hair follicle tissue.

4. The method according to any of claim 1 or 2, wherein the androgen non-inhibited cells are androgen stimulated cells, for example wherein the hair follicle tissue is beard hair follicle tissue, chest hair follicle tissue, axilla hair follicle tissue and/or pubic hair follicle tissue.

5. The method according to any of the preceding claim, wherein the androgen non-inhibited cells comprise or consist of dermal papilla (DP) cells.

6. The method according to any of the preceding claims, in which the androgen non-inhibited cells implanted in step (3) of claim 1 rejuvenate the miniaturised and/or miniaturising hair follicles by reactivating androgen non-inhibited cells of the miniaturised and/or miniaturising hair follicles, and/or replacing androgen non-inhibited cells in the miniaturised and/or miniaturising hair follicles.

7. The method according to any of the preceding claims, in which the hair follicle tissue is obtained from a body region known or expected to comprise androgen non-inhibited cells.

8. The method according to any of the preceding claims, in which the hair follicle tissue is extracted mechanically, for example using follicle unit extraction (FUE).

9. The method according to any of the preceding claims, in which obtaining androgen non-inhibited cells from hair follicle tissue comprises obtaining androgen non-inhibited cells from a suspension of hair follicle tissue cells by antibody-assisted selection.

10. The method according to any of the preceding claims, wherein the step of culturing the androgen non-inhibited cells expands the number of androgen non-inhibited cells by at least about 50- to 100-fold, for example by at least about 500- to 1000-fold.

11. The method according to any of the preceding claims, in which the level of expression of one or two or more genes is used as a biomarker of androgen insensitivity to identify and/or select androgen non-inhibited cells, wherein the one or two or more genes is selected from the group consisting of STX17 antisense RNA 1; prostaglandin 12 (prostacyclin) synthase; calmegin; glypican 6; integrin beta 8; collagen, type X, alpha 1; reelin; carbonic anhydrase XIII; DEP domain containing MTOR-interacting protein; and HAUS augmin like complex subunit 6.

12. The method according to claim 11, in which steps (1) and/or (2) of claim 1 comprise selecting or sorting the cells for androgen non-inhibited cells using the biomarker.

13. The method according to any of the preceding claims, in which the androgen is dihydrotestosterone (DHT).

14. The method according to any of the preceding claims, in which the hair follicles and/or androgen non-inhibited cells are human.

15. The method according to any of the preceding claims, in which the androgen non-inhibited cells are obtained from a subject in step (1) of claim 1 and after culturing in step (2) of claim 1 implanted into same subject in step (3) of claim 1.

16. A composition comprising an in vitro-expanded population of androgen non-inhibited cells for rejuvenating hair.

17. The composition according to claim 16, in which the androgen non-inhibited cells are as defined in any of claims 1 to 15.

18. The composition according to either of claim 16 or claim 17, where cells are from autologous or allogeneic origin.

19. The composition according to any of claims 16 to 18, formulated for injection.

20. A composition as defined in any of claims 16 to 19 for use in a method of treatment.

21. A composition as defined in any of claims 16 to 19 for use in a method for the rejuvenation of hair growth and/or for the retardation of hair loss.

22. Use of a composition as defined in any of claims 16 to 19 in the manufacture of a medicament for the rejuvenation of hair growth and/or for the retardation of hair loss.

23. The composition for use of claim 20 or 21, or the use of claim 22, for application to a subject having alopecia, such as androgenic alopecia.

24. The composition for use of claim 20, 21 or 23, or the use of claim 22 or 23, in which the method or use is entirely cosmetic.

25. The composition for use of claim 20, 21, 23 or 24, or the use of claims 22 to 24, in which the composition is administered by injection, for example by a non-physician medical technician.

26. The use of the composition as defined in any of claims 16 to 19 in a system for analysing hair follicle cells and/or for testing a cosmetic or pharmaceutical agent.
